(19) United States
(12) Patent Application Publication
(10) Pub. No.: US 2011/0196493 A1
(43) Pub. Date: Aug. 11, 2011

(54) CAGE SYSTEM TO ANTERIOR LUMBAR INTERVERTEBRAL FUSION

(76) Inventor: Luiz Henrique Mattos Pimenta, Sao Paulo (BR)

(21) Appl. No.: 12/760,232

(22) Filed: Apr. 14, 2010

(30) Foreign Application Priority Data
Feb. 11, 2010 (BR) 018100004584

(51) Int. Cl. A61F 2/44

(52) U.S. Cl. 623/17.16

(57) ABSTRACT

An implant for the human body, and in particular to an implant suitable for placement between two adjacent vertebrae of the spine, the implant having a cage to allow for bone growth in and around the cage.

### FIELD OF THE INVENTION

The present invention relates to an implant for the human body, and in particular to an implant suitable for placement between two adjacent vertebrae of the spine, the implant having a cage to allow for bone growth in and around the cage.

### BACKGROUND OF THE INVENTION

An intervertebral implant is surgically implanted in a person's spine between adjacent vertebrae to treat and correct one or more abnormalities in the spine. More particularly, intervertebral implants can be used to provide support for adjacent vertebrae. They also provide and maintain proper spacing between the adjacent vertebrae. A spinal abnormality may be the result of one or more different causes, including degenerative vertebral disorders, diseases, infections, or traumas. Furthermore, spinal implants are used in many different surgical procedures and courses of treatment, including in arthrodesis, and in the correction of lumbar support instabilities, spondylolisthesis, discopathies, damage caused by trauma or tumors, to name a few.

Some intervertebral implants are employed to achieve vertebral fixation, in which two or more adjacent vertebrae are anchored to one another. A primary purpose of vertebral fixation is to reduce or eliminate motion between the two vertebrae. Other implants are used in a vertebral fusion procedure in which the growth of bone tissue is promoted between adjacent vertebrae to eliminate motion between the vertebrae. In such a procedure, the implant is typically used to immobilize the adjacent vertebrae while the bone fusion progresses. The implant may also be adapted for bone growth around or through the implant. The implant may also have means for receiving bone graft or bone substitute, which can be positioned on or in the implant prior to implantation.

An example of an existing implant is the STALIF TT™ device, made by Surgicraft of the United Kingdom. This device comprises a body having four holes defined therein to receive retaining screws. When the device is implanted into the spine, the implant body is positioned between adjacent vertebrae and two screws are screwed into the upper vertebra through two of the receiving holes, and two screws are screwed into the lower vertebra through the other two receiving holes. Although the end results achieved by the STALIF TT device may be satisfactory, these results are only achieved after a lengthy healing and recovery time. In addition, the resultant stability is achieved after the growth of bone around the device, and the rate of this bone growth is generally slower than what is desired. The lengthy healing and bone growth time results in a long period of pain and reduced mobility for the patient.

For the foregoing reasons, it can be appreciated that a need exists for an intervertebral implant that achieves a faster healing and recovery time, faster rates of bone growth and fusion, and reduced chances of instability.

### SUMMARY OF THE INVENTION

The present disclosure provides an intervertebral implant.

In one aspect, the present disclosure is directed to an intervertebral implant to be positioned between adjacent first and second vertebrae in a spine, the implant comprising: (a) a fusion cage comprising: a top side, engageable with the first vertebra; a bottom side, opposite the top side, the bottom side engageable with the second vertebrae, the cage defining at least one primary opening extending from the top side to the bottom side through the cage to allow for the growth of bone in the opening; first and second opposing lateral sides; an anterior side, the anterior side being the first side of the fusion cage to be inserted into the spine; and a posterior side opposite the anterior side, the cage defining a first screw aperture extending from the posterior side at a downwardly angle through the cage to the bottom side, the cage defining a second screw aperture extending from the posterior side at an upwardly angle through the cage to the top side; (b) a first screw having a head and a shank for securing the fusion cage to the first vertebra by inserting the shank into the first screw aperture from the posterior side of the aperture and screwing the shank into the first vertebra; and (c) a second screw having a head and a shank for securing the fusion cage to the second vertebra by inserting the shank into the second screw aperture from the posterior side of the aperture and screwing the shank into the second vertebra.

### BRIEF DESCRIPTION OF THE DRAWINGS

The present disclosure will be better understood having regard to the drawings.

FIG. 1 is a top view of one embodiment of the intervertebral implant.

FIG. 2 is a cross-sectional side view taken along the line 2-2 of FIG. 1.

FIG. 3 is a front view of the embodiment shown in FIG. 1.

FIG. 4 is a rear view of the embodiment shown in FIG. 1.

FIG. 5 is a perspective view of the embodiment shown in FIG. 1.

FIG. 6 is a side view of one embodiment of an anchoring screw to be used with the intervertebral implant.

FIG. 7 is a cross-sectional view taken along the line 7-7 of FIG. 6.

FIG. 8 is a top view of the anchoring screw shown in FIG. 6 showing the head of the screw.

FIG. 9 is a perspective view of the embodiment shown in FIG. 6.

FIG. 10 is a bottom perspective view of the embodiment of the intervertebral implant shown in FIG. 1 with anchoring screws installed.

FIG. 11 is a top perspective view of the embodiment of the intervertebral implant shown in FIG. 1 with anchoring screws installed.

### DETAILED DESCRIPTION OF THE INVENTION

The present intervertebral implant is described in one embodiment in the following disclosure with reference to the Figures. While this embodiment is described in the context of an intervertebral implant suitable for implantation in the lumbar region of the spine of a human, the scope of the present disclosure is not intended to be limited to lumbar implants. The present intervertebral implant can be used in other regions of the human spine and in other animals.

The various features and components of the present intervertebral implant are now described with reference to the Figures.

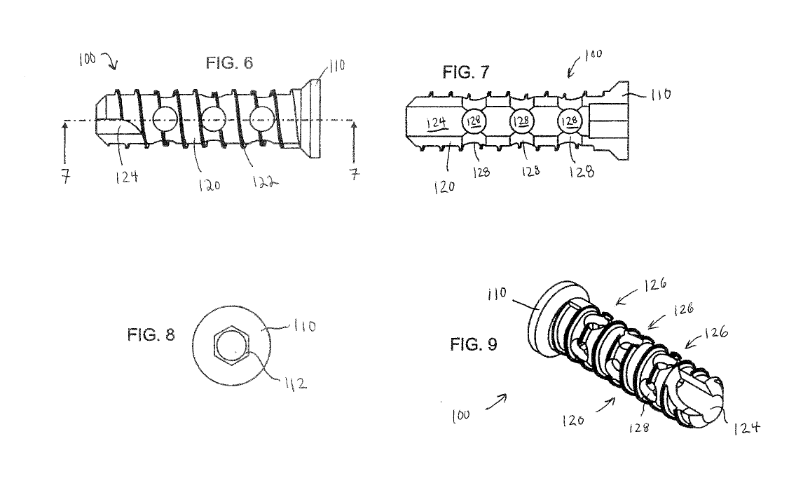

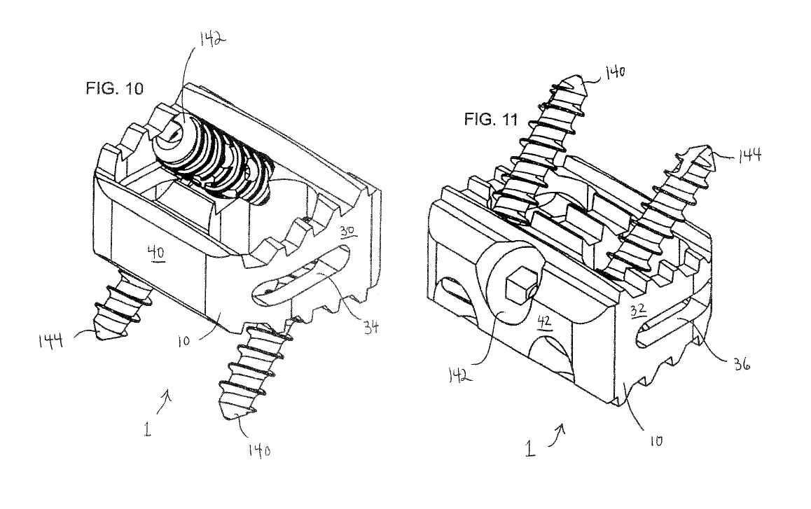

FIGS. 10 and 11 show one embodiment of the intervertebral implant 1, which generally comprises a fusion cage 10 and at least first and second screws 140 and 142, respectively. FIGS. 1 to 5 show different views of one embodiment of the fusion cage 10. FIGS. 6 to 9 show different views of one type of screw that may be used with the intervertebral implant 1.

With reference now to FIGS. 1, 3 and 5, in at least one embodiment of the implant, fusion cage 10 comprises a top side 20, a bottom side 22 opposite the top side, first and second lateral sides 30 and 32, respectively, an anterior side 40 and a posterior side 42. Anterior side 40 and posterior side 42 are shown in FIGS. 3 and 4, respectively. In at least one embodiment, cage 10 is dimensioned to be similar in size to a human lumbar vertebra.

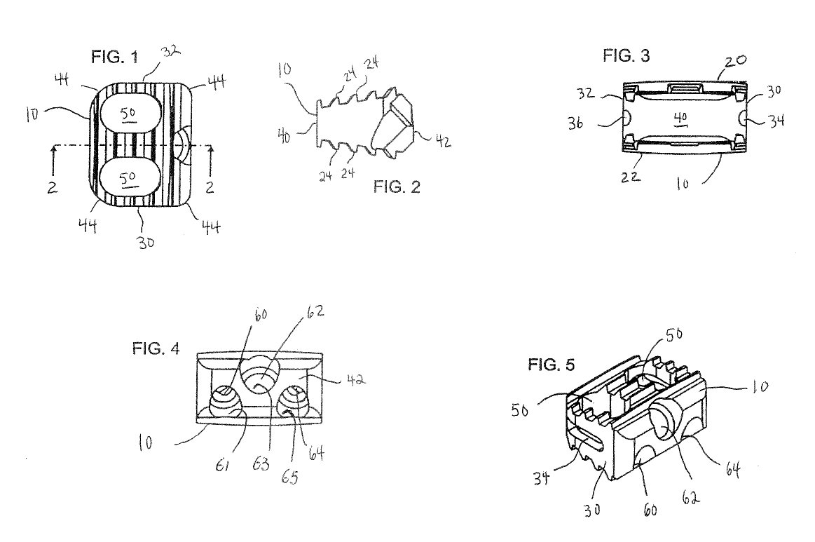

Top side 20 is adapted for engagement with a first vertebra, whereas bottom side 20 is adapted for engagement with a second vertebra. One or more of the corner edges 44 of cage 10 can be curved or rounded, as shown in FIG. 1. Furthermore, the implant is generally to be inserted into a spine with anterior side 40 being the leading side. As shown in FIG. 2, in at least one embodiment the thickness of cage 10 can be slightly tapered from posterior side 42 to anterior side 40. In addition, top side 20, bottom side 22, or both can each have one or more teeth 24 for improving the frictional engagement of cage 10 with the adjacent vertebra or vertebrae.

As shown in FIGS. 1 and 5, cage 10 defines at least one primary opening 50 for allowing bone growth therein. In at least one embodiment, primary opening 50 can extend through cage 10 between top side 20 and bottom side 22. Furthermore, in at least one embodiment cage 10 can define two primary openings 50. As best shown in FIG. 1, in one embodiment the two primary openings 50 can be symmetrically formed in cage 10. More specifically, the two openings 50 can be formed on opposing sides of an imaginary plane that bisects cage 10 in a vertical orientation between anterior 40 and posterior 42 sides of cage 10. In addition, as best shown in FIGS. 3, 10 and 11, the cage can also define first and second lateral openings 34 and 36, respectively. First lateral opening 34 can extend through cage 10 from first lateral side 30 to a one of the at least one primary opening 50. Likewise, second lateral opening 36 can extend through cage 10 from second lateral side 32 to a one of the at least one primary opening 50.

After implantation of the implant into the spine, the growth of bone from adjacent vertebrae, from a bone graft or from bone substitute positioned in or proximate the implant by a surgeon can grow in and around primary opening 50 to fuse cage 10 to one or both of the adjacent vertebrae.

Cage 10 also comprises at least a first screw aperture 60 and a second screw aperture 62, which are shown in FIGS. 4 and 5. The embodiment shown in the Figures also has an optional third screw aperture 64. First screw aperture 60 extends through cage 10 from posterior side 42 at an upward angle to top side 20 of the cage. Second screw aperture 62 extends through cage 10 from posterior side 42 at a downward angle to bottom side 22 of the cage. In at least one embodiment, the optional third screw aperture 64 extends through cage 10 from posterior side 42 at an upward angle to top side 20 of the cage. Furthermore, as shown in the Figures, in at least one embodiment having the optional third screw aperture 64, first and third screw apertures 60 and 64, respectively, can be formed on opposite sides of second screw aperture 62.

In addition, cage 10 can define a recess in posterior side 42 of the cage concentric with one or more of screw apertures 60, 62 and 64. The embodiment shown in FIGS. 4 and 5 comprises first, second and third recess 61, 63 and 65 at first, second and third screw apertures 60, 62 and 64, respectively. Each recess can be shaped and dimensioned to receive the head of a screw, which is described in further detail below.

The cage itself can be made of titanium or any other suitable material known in the art.

As described above, implant 1 further comprises at least a first screw 140 and a second screw 142 for securing cage 10 to first (upper) and second (lower) vertebrae, respectfully. These screws provide a primary fixation system in which cage 10 is immediately and directly anchored to the first and second vertebrae. The implant 1 shown in the FIGS. 10 and 11 further comprises an optional third screw 144 for further securing cage 10 to the first (upper) vertebra. The optional third screw 144 is receivable into the optional third screw aperture 64. It is to be appreciated that implant 1 could comprise one or more additional screw apertures along with one or more additional screws.

In at least one embodiment, the diameters of the screws can differ. For example, in the embodiment illustrated in the Figures, the diameter of second screw 142 is larger than the diameters of first and third screws 140, 144. This may be desirable as second screw 142 is the only screw that is to connect cage 10 to the second (lower) vertebra. A screw having a larger diameter may provide a stronger connection of cage 10 to the second (lower) vertebra. Of course, the diameter of the second screw aperture 62 would need to be sufficient in order to receive the larger second screw 142.

The screws of the implant can be of any suitable type known in the art. Each screw has a head and a shank, the shank having a thread. In at least one embodiment, one or more of the screws can be of type shown in FIGS. 6 to 9. Screw 100 has a head 110 and a shank 120, the shank having a thread 122. Screw head 110 can define a hexagonal cavity for receiving a tool for screwing the screw (see FIG. 8). However, it will be appreciated that head 110 could also have a cavity or a protrusion of a different shape suitable for receiving a different tool. Shank 120 defines a hollow core 124 and a plurality of holes 128 extending radially therefrom. Hollow core 124 and holes 128 allow for the growth of bone around and in these openings in screw 100, thereby allowing for a more complete fusion of bone to the implant 1. In at least one embodiment, shank 120 defines one or more “sets” of holes 126 (see FIG. 9). Each set of holes has a plurality of holes 128 substantially aligned in a single plane orthogonal to the longitudinal axis of shank 120. Furthermore, the holes 128 of a set 126 can be substantially equally spaced around the circumference of shank 120. As shown in FIG. 9, in at least one embodiment, a set 126 has four holes 128 spaced substantially at ninety-degree intervals around the circumference of shank 120. Shank 120 can define one or more sets 126 along its length.

Similarly to cage 10, the screws of implant 1 can be made of titanium or any other suitable material known in the art.

The implantation of the implant will now be described. This process will be described in relation to the particular embodiment shown in FIGS. 1 to 5, 10 and 11. A surgeon will first insert the implant between adjacent first and second vertebrae in the spine. The implant may be implanted in the lumbar region of the spine, or in another region of the spine. The surgeon will then connect and secure cage 10 to both first and second vertebrae using first, second and third screws 140, 142 and 144. Shanks 120 of the screws will be inserted through the screw apertures 60, 62 and 64 from the posterior side of cage 10. Screws 140, 142 and 144 will be fully screwed into the adjacent first and second vertebrae. FIGS. 10 and 11 show implant 1 with the screws fully inserted into their respective screw apertures. Once the implant has been implanted, there is the expectation, or at least the hope that there will be bone growth around and into the implant, thereby forming a rigid and solid piece between adjacent vertebrae.

The resulting bone growth can be promoted by various means, which are known in the art. For example, an inductor substance can be used to activate the growth of bone in the patient in the desired area. Furthermore, the bone growth can be autologous or heterologous.

The previous detailed description is provided to enable any person skilled in the art to make or use the present invention. Various modifications to those embodiments will be readily apparent to those skilled in the art, and the generic principles defined herein may be applied to other embodiments without departing from the spirit or scope of the invention described herein. Thus, the present invention is not intended to be limited to the embodiments shown herein, but is to be accorded the full scope consistent with the claims, wherein reference to an element in the singular, such as by use of the article “a” or “an” is not intended to mean “one and only one” unless specifically so stated, but rather “one or more”. All structural and functional equivalents to the elements of the various embodiments described throughout the disclosure that are known or later come to be known to those of ordinary skill in the art are intended to be encompassed by the elements of the claims. Moreover, nothing disclosed herein is intended to be dedicated to the public regardless of whether such disclosure is explicitly recited in the claims.

What is claimed:

1. A intervertebral implant to be positioned between adjacent first and second vertebrae in a spine, the implant comprising:
(a) a fusion cage comprising:
a top side, engageable with the first vertebra;
a bottom side, opposite the top side, the bottom side engageable with the second vertebrae, the cage defining at least one primary opening extending from the top side to the bottom side through the cage to allow for the growth of bone in the opening;
first and second opposing lateral sides;
an anterior side, the anterior side being the first side of the fusion cage to be inserted into the spine; and
a posterior side opposite the anterior side, the cage defining a first screw aperture extending from the posterior side at a downwardly angle through the cage to the bottom side, the cage defining a second screw aperture extending from the posterior side at an upwardly angle through the cage to the top side;
(b) a first screw having a head and a shank for securing the fusion cage to the first vertebra by inserting the shank into the first screw aperture from the posterior side of the aperture and screwing the shank into the first vertebra; and
(c) a second screw having a head and a shank for securing the fusion cage to the second vertebra by inserting the shank into the second screw aperture from the posterior side of the aperture and screwing the shank into the second vertebra.

2. The intervertebral implant of claim 1 wherein the fusion cage further defines a third screw aperture extending from the posterior side at an upwardly angle through the cage to the top side, and the implant further comprises a third screw having a head and a shank for further securing the fusion cage to the second vertebra by inserting the shank into the third screw aperture from the posterior side of the aperture and screwing the shank into the second vertebra.

3. The intervertebral implant of claim 2 wherein the first and third screw apertures are located on opposite sides of the second screw aperture.

4. The intervertebral implant of claim 1 wherein the fusion cage further defines:
(d) a first lateral opening extending from the first lateral side through the cage to a one of the at least one primary opening; and
(e) a second lateral opening extending from the second lateral side through the cage to a one of the at least one primary opening.

5. The intervertebral implant of claim 1 wherein at least one of the top and bottom sides comprises a plurality of teeth for improving the frictional engagement of the cage with at least one vertebrae.

6. The intervertebral implant of claim 1 wherein the cage defines two primary openings, the openings being on opposing sides of a plane bisecting the cage in a vertical orientation between the anterior and posterior sides of the cage.

7. The intervertebral implant of claim 2 wherein the fusion cage further defines first, second and third recesses in the posterior side of the cage concentric with the first, second and third apertures, respectively, the first, second and third recesses shaped to receive a head of a screw therein.

8. The intervertebral implant of claim 2 having first, second and third screws the shanks of which being receivable into the first, second and third screw apertures, respectively.

9. The intervertebral implant of claim 2 wherein the first screw aperture has a larger diameter than the diameters of the second and third screw apertures.

10. The intervertebral implant of claim 9 having first, second and third screws the shanks of which being receivable into the first, second and third screw apertures, respectively, the diameter of the shank of the first screw being larger than the diameters of the shanks of the second and third screws, respectively.

11. The intervertebral implant of claim 1 wherein at least part of the shank of the at least one screw defines a hollow core and a plurality of holes extending radially from the hollow core through the shank.

12. The intervertebral implant of claim 1 wherein the shank of the at least one screw defines a hollow core and at least one set of holes, the holes of the at least one set extending radially from the hollow core through the shank, being aligned about a single plane orthogonal to the longitudinal axis of the shank, and being substantially equally spaced around the circumference of the shank.

13. The intervertebral implant of claim 12 wherein the shank comprises a plurality of sets of holes along the length of the shank.

14. The intervertebral implant of claim 12 wherein the at least one set of holes consists of four holes spaced substantially at 90 degree intervals around the circumference of the shank.

15. The intervertebral implant of claim 14 wherein the shank has three sets of holes along the length of the shank.
